(19) United States
(12) Patent Application Publication
(10) Pub. No.: US 2015/0164484 A1
(43) Pub. Date: Jun. 18, 2015

(54) Methods and kit system for assessing fertility with vaginal CA125/MUC16 levels

(71) Applicants: Stephen J. Usala, Amarillo, TX (US); William C. Biggs, Amarillo, TX (US); Faye O'Brien-Usala, Amarillo, TX (US)

(72) Inventors: Stephen J. Usala, Amarillo, TX (US); William C. Biggs, Amarillo, TX (US); Faye O'Brien-Usala, Amarillo, TX (US)

(21) Appl. No.: 14/133,531

(22) Filed: Dec. 18, 2013

(51) Int. Cl. A61B 10/00; A61B 10/02; A61B 5/00; A61F 13/38

(52) U.S. Cl. CPC A61B 10/0012; A61F 13/38; A61B 10/0045; A61B 10/02; A61B 5/486; A61B 2010/0074

(57) ABSTRACT

Disclosed are methods for assessing the period of potential fertility, days of maximum fertility, and transition to the infertile phase via measurement and processing of vaginal CA125 (MUC16) levels. In one embodiment, the fertile and infertile phases of the ovulatory normal female menstrual cycle are defined by a pattern or signature of day-specific vaginal CA125 levels. Suitably, signatures are based upon daily cotton swab collection of vaginal epithelial and fluid plus subsequent assay of CA125. Day-specific vaginal CA125 assay levels increase during the period of potential fertility and reach a maximum with inflection point generally before and near the time of ovulation. Derived functions of day-specific vaginal CA125 are used to predict fertility, enhance conception, and for birth control.

### CROSS-REFERENCE TO RELATED APPLICATIONS

Not applicable.

### STATEMENT REGARDING FEDERALLY SPONSORED RESEARCH OR DEVELOPMENT

Not applicable.

### BACKGROUND OF INVENTION

1. Field of the Invention

The subject matter of this disclosure is in the field of apparatus and methods for assessing female mammalian fertility levels.

2. Background of the Invention

Natural family planning involves pregnancy avoidance or achievement by natural means and is practiced by many male-female couples. For natural contraception, the likelihood of avoiding pregnancy can be increased via periodic abstinence wherein intercourse is limited to naturally infertile portions of a female mammal's menstrual cycle, during pregnancy, and after menopause. Conversely, the likelihood of pregnancy achievement can be increased via increased sexual activity during highly fertile portions of the menstrual cycle. Female fertility is highest over a six day window, namely, the day of ovulation and the preceding five days (this time period is sometimes referred to as the “period of potential fertility”). Within this period of potential fertility, the days of maximum fertility are most frequently the actual day of ovulation and the preceding day. In view of the foregoing, it comes as no surprise that fertility prediction methodologies are needed for successful natural family planning.

One common type of natural contraception is periodic abstinence by calendar rhythm, which relies on predictions of a female's infertility or fertility based on the historical timing and duration of her previous menstrual cycles. However, the National Health Statistics Report recently reported the probability of having an unintended pregnancy while typically practicing periodic abstinence by calendar rhythm is over twenty percent. One cause of the failure of periodic abstinence in preventing pregnancy may be that historical timing and duration of previous menstrual cycles may not be the most accurate, reliable, or timely indications of fertility. Thus, accurate and real-time fertility prediction methodologies are still needed for improved natural family planning.

Observational forms of fertility prediction methods also exist. For example, visual observation of cervical mucus, cervical-vaginal secretions, or vaginal moisture have been used for assessing fertility. While capable of predicting fertility, these observational methods are not entirely suitable for all situations because the observations are subjective and frequently may require a “double-check” by another indicator, such as basal body temperature, with limited gains in accuracy.

The need for accurate and reliable fertility predictions is further established in the background of U.S. Pat. No. 7,722,547 (issued May 25, 2010) which went on to disclose apparatus and methods for real-time fertility assessments based on die swells of vaginal fluid (i.e., cervical-vaginal fluid). A similar method, formerly on the market as Rovumeter™, is a fertility prediction based on volumetric and/or gravimetric changes in cervical-vaginal fluid disclosed in U.S. Pat. No. 4,534,362 (issued Aug. 7, 1985). These methods, while improvements to other fertility predictions, are limited by the difficulty of capturing sufficient volumes of vaginal fluid samples (typically greater than 0.2 ml) for making the fertility assessment. Thus, a need still exists for accurate and reliable fertility predictions that do not require large quantities of vaginal fluid.

Further technology to predict the period of potential fertility involves monitoring the urinary estrone-3-glucuronide (E3G) and/or luteinizing hormone (LH). Although presently available as a kit and capable of predicting the day of ovulation, these methods of natural birth control have high failure rates because: (a) accurate predictions of the probability of conception on any given day are dependent on both (i) the day's temporal proximity to the actual day of ovulation and (ii) the fecundity (conduciveness to sperm transport) of the woman's cervical-vaginal mucus or fluid; and (b) urinary E3G and LH do not necessarily correlate with the fecundity of cervical-vaginal fluid (Note: within the fertile window, the type or fecundity of mucus observed on the day of intercourse is more predictive of conception than the timing relative to ovulation). Therefore, a need exists for fertility prediction or assessment methods that account for the day-specific fecundity of a woman's cervical-vaginal fluid as well as temporal proximity to the day of ovulation.

Cervical mucus includes O-glycylated proteins, called mucins, and increased levels of the supposedly dominant mucin, MUCSB, are observed in the middle of the menstrual cycle. Other mucins, MUC5AC, MUC6, MUC1, and MUC16, are also known to be present in cervical mucus, but their role in reproduction and fertility is unknown. Importantly, other than this disclosure, presently no methods or apparatus exist for fertility assessment based on mucin of any type.

MUC16 (also known as CA125) is a tumor antigen and its level in serum is primarily used to detect and monitor the growth of ovarian cancer. CA 125 serum levels also have a controversial role in detecting the presence of endometriosis. MUC16 may also play a role in assessing cancer invasiveness, immune function and trophoblast implantation in the uterus. Although useful for cancer diagnosis, the scientific literature generally teaches away from CA125 as a factor in sperm transport or its being an important constituent of cervical mucus; for example it has been found that CA125/MUC16 is dispensable for mouse development and reproduction. Furthermore, serum CA125 levels have not been found to be useful in the clinical management of in vitro fertilization.

Serum CA125 levels, although perhaps being mildly elevated at menses, do not track the ovulatory cycle. In addition, recent literature on CA125 in cervical mucus and cervical-vaginal fluid teaches away from any method using CA125 to monitor the phase of the menstrual cycle. For instance, one study, while reporting high levels of CA125 in cervical mucus (173,900±128,000 U/ml), found that cervical mucus CA125 levels were “relatively constant along the cycle” and “no clear trend could be detected related to hormonal changes and ovulation” (Martinez A R, Thomas C M, Segers M F, et al., CA-125 levels in cervical mucus during the menstrual cycle. Fertil Steril 61:843-849, 1994). Another study (He S M, Xing F Q, Sui H, et al. CA125 expression in cervical and vaginal secretions in women in normal reproductive period. Nan Fang Yi Ke Da Xue Xue Bao 30:173-5, 2010) reported levels of CA125 in swabs of vaginal secretions during the “normal reproductive period” without identifying any cyclic changes in the CA125 levels in cervical mucus or vaginal fluid. Furthermore, in another study (He S M, Xing F, Sui H, et al. Determination of CA125 levels in the serum, cervical and vaginal secretions, and endometrium in Chinese women with precancerous disease or endometrial cancer. Med Sci Monit 17:CR618-625, 2011) no statistical differences in CA125 levels between menstrual cycle phases were shown in swabs of cervical-vaginal fluid.

In summary, known fertility prediction or assessment methods are not entirely suitable for natural family planning. Many of the known methods of predicting a woman's fertility on a given day rely too heavily on an indicator occurrence relative to the day of ovulation and, at the same time, fail to account for the fecundity of the woman's vaginal fluid. Other methods require excessively large amounts of vaginal fluid for making fertility assessments. Scientific and patent literature teach away from using the mucin and tumor marker, MUC16/CA125, in fertility assessments. In other words, despite many attempts at a solution, a need still exists for fertility assessment or prediction methodologies without the drawbacks associated with known methods.

### SUMMARY OF THE INVENTION

It is an objective of this disclosure to describe fertility assessment or prediction methodologies that are accurate and reliable without the drawbacks of known fertility assessments or predictions. In one embodiment, the fertility assessment method correlates CA125/MUC16, with both (a) the time of ovulation and (b) the fecundity of cervical-vaginal secretions. This disclosure represents an objective, quantitative, sensitive, precise, reliable, economically feasible methodology for signaling the beginning of the fertile phase of the menstrual cycle for natural family planning.

It is another objective of the present disclosure and various embodiments described herein to provide an improved method for generally assessing the period of potential fertility and time-of-cycle in human females for the timing sexual intercourse for enhancement of the probability of conception, contraception, or for gynecologic and reproductive diagnosis. It is yet another object of this disclosure to provide a method capable of utilizing the CA125 (MUC16) concentrations from small quantities of vaginal cells and fluid to obtain meaningful information on the phase and health of the female menstrual cycle. It is a further object of this description to provide a method which establishes a normal pattern or signature of vaginal CA125 levels relative to the day of ovulation (i.e., day-specific vaginal CA125 levels), which pattern corresponds to the phase of the ovulatory cycle in the normal female. It is still another objective of this disclosure to describe a method of using a day-specific vaginal CA125 signature to signal the period of potential fertility and the transition to the luteal (post-ovulatory or infertile) phase in ovulatory normal women for the purposes of family planning. It is a further object of this description to disclose a method which serves to diagnosis reproductive and gynecological pathology utilizing perturbations of the day-specific vaginal CA125 signature of the ovulatory, fertile, normal menstrual cycle in human females. Finally, it is an object of the present application and various embodiments described herein to provide a method of assessing female fertility levels, either as a stand-alone technology or in conjunction with other fertility technologies, by which a woman could examine herself with little outside assistance in the form of a test kit.

In one embodiment, disclosed is a method of using a test kit for assessing the fertility of a female subject via measurement of CA125/MUC16 levels. According to this method, fertility is assessed via:
(1) establishing a normal pattern of vaginal CA125/MUC16 levels over a typical ovulatory cycle (e.g., from a study of multiple women), which is used to determine day-specific vaginal CA125/MUC16 levels as a function of time-of-cycle;
(2) collecting a swab of vaginal epithelial and fluid (distinct from pure cervical mucus) from the distal vagina near the vaginal orifice of the subject;
(3) measuring the CA125/MUC16 level in the swab provided in a kit; and
(4) determining the time-of-cycle (fertile phase versus maximum fertility day(s) versus infertile/post-ovulatory phase) using the established function or signature of day-specific vaginal CA125/MUC16 levels.
   Suitably, the methodology may be used to determine the time of cycle including the beginning of the period of potential fertility of the subject's ovulatory cycle and the transition to the post-ovulatory/luteal infertile phase of the ovulatory cycle. In a preferred embodiment, the methodology is useful for timing the subject's sexual intercourse schedule to enhance fertility or as a form of birth control. In an alternate embodiment, the method can be used for diagnosing endocrine disease or ovarian dysfunction vis-à-vis perturbations of the subject's vaginal CA125/MUC16 cyclic levels when compared to the normal pattern.

Other objectives of the application and various embodiments described herein will become apparent to those skilled in the art once the preferred embodiments have been shown and described. These objectives are not to be construed as limitations of the applicant's invention, but are merely aimed to suggest some of the many benefits that may be realized by the apparatus and methods of the present application and with its many embodiments which may be also deployed in a convenient kit for the user.

Throughout this invention disclosure, ‘CA125’ will be used interchangeably with ‘MUC16’ since they are the same molecule. At times ‘CA125/MUC16’ will be used to emphasize the mucinous characteristics and biology of CA125.

Numerous antibodies have been generated against CA125. These antibodies are classified as OC125-like (group A), M11-like (group B) or OV197 (group C) depending on the antigenic determinant. The monoclonal antibody used in the immunoassay (OM-MA™ Siemans) for this disclosure is OV185, which detects the M11 epitope (Sibley PEC, BR-MA, GI-MA and OM-MA: immunoassays for the tumor markers CA15-3, CA19-9 and CA125. Siemans Medical Solutions™. http://www.medical.siemens.com/siemens/en_GLOBAUL/gg_diag_FBAs/files/medical/cancer/tech_reports/ZB139-A.pdf). It is important to note, that the disclosed methodology is not limited to detection of CA125 with OV185.

### BRIEF DESCRIPTION OF THE FIGURES

The manner in which these objectives and other desirable characteristics can be obtained is explained in the following description and attached figures in which:

FIGS. 1A, 1B, and 1C are x-y plots of vaginal CA125 levels during short, medium, and long ovulatory cycles for normal women;

FIG. 2 is the mean or average day-specific vaginal CA125 signature for thirteen menstrual cycles of ten different normal ovulatory women;

FIG. 3 is a processed mean day-specific vaginal CA 125 function or signature for the thirteen menstrual cycles of FIG. 2. Here the day-specific vaginal CA125 levels for each cycle were normalized to (divided by) the minimum vaginal CA125 level for the first and second days of swab sampling;

FIGS. 4A, 4B, and 4C are an example of the use of the signature of FIG. 3 to signal the start of the period of potential fertility for subject cycle 14-1 (FIG. 4A). In FIGS. 4B and 4C the day-specific CA125 levels are normalized to the minimum vaginal CA125 level for the first and second days of swab sampling;

FIG. 5 shows the abnormal day-specific vaginal CA125 profile for an anovulatory cycle of a perimenopausal woman;

FIG. 6 is a chart of the vaginal CA125 levels of a menopausal woman; and,

FIG. 7 is a table of fertility metrics using the disclosed method from ten ovulatory normal woman compared with conventional natural family planning methods.

### DETAILED DESCRIPTION OF THE INVENTION

Generally disclosed is a method of assessing the fertility of a female subject via measurement of vaginal CA125/MUC16 levels. According to a preferred embodiment method, fertility is assessed via:
(1) establishing a normal pattern of vaginal CA125/MUC16 levels over a typical ovulatory cycle (e.g., from a study of multiple women) (This is to determine the day-specific levels of vaginal CA125/MUC16 as a function of time-of-cycle as indexed to an indicator of the day of ovulation);
(2) collecting a swab of vaginal epithelial and fluid from the distal vagina near the vaginal orifice (distinct from cervical mucus) of the subject;
(3) measuring the CA125/MUC16 level in the swab; and
(4) determining the time-of-cycle (period of potential fertility versus days of maximum fertility versus infertile luteal days), based on the measured vaginal CA125 level and knowledge of the normal vaginal CA125 signature.

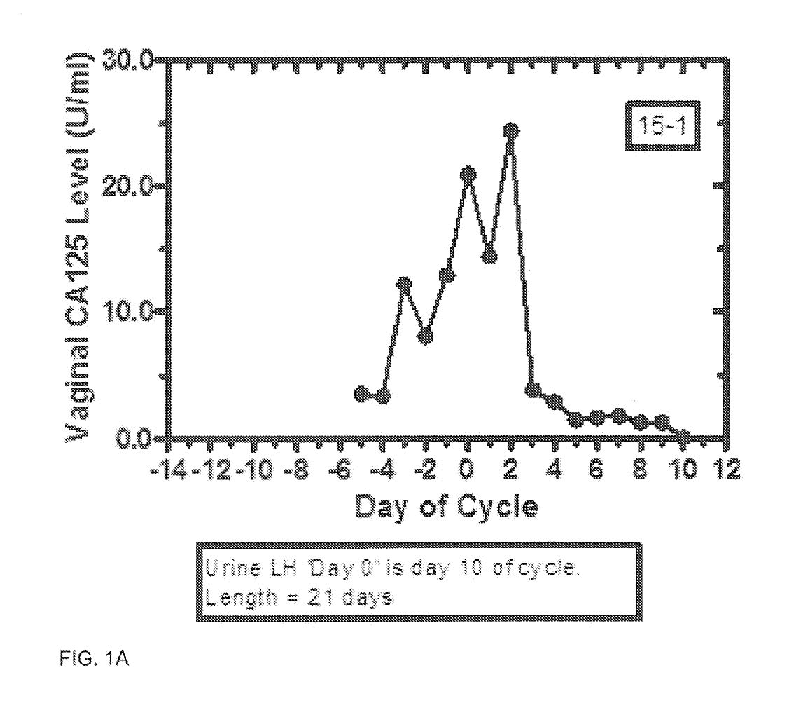

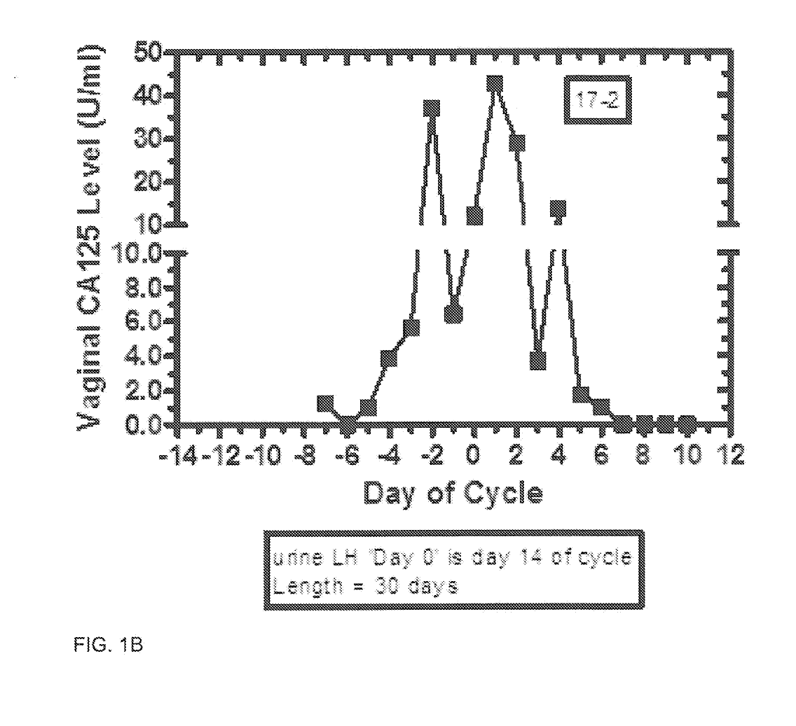

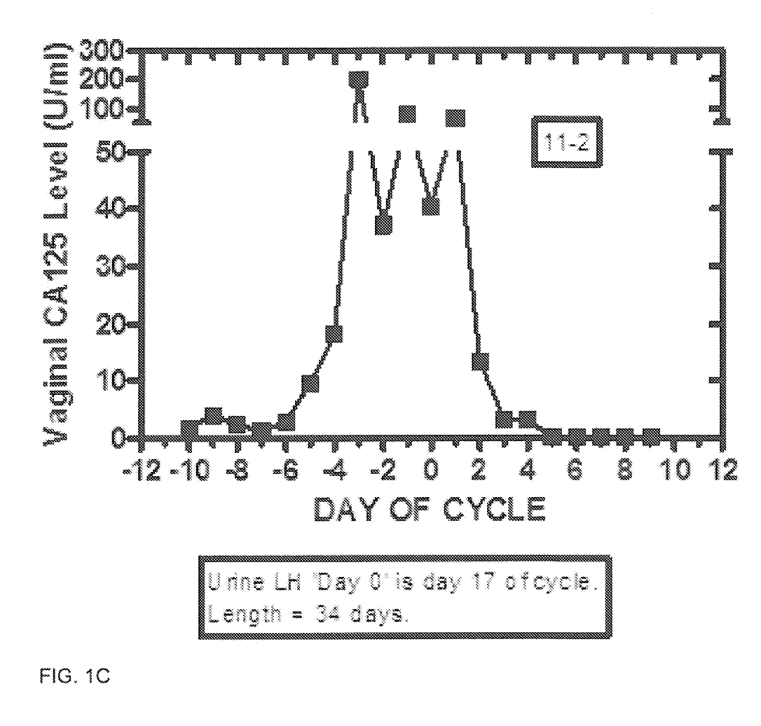

FIGS. 1A through C are useful for illustrating the step of establishing a normal pattern of vaginal CA125/MUC16 levels over a typical ovulatory cycle. Specifically, FIGS. 1A through 1C depict x-y plots of vaginal CA125 level (in U/ml) versus day of the menstruation cycle for three subjects: PT15-1 (FIG. 1A); PT17-2 (FIG. 1B); and PT11-2 (FIG. 1C). In the plots, day zero (0) in the x-axis is the day of positive urine luteinizing hormone (uLH), which is a signal for the day of ovulation. For PT15-1 (FIG. 1A), day zero (0) on the x-axis is the tenth day of a twenty-one day menstrual cycle (short menstrual cycle). For PT17-2 (FIG. 1B), day zero (0) on the x-axis represents the fourteenth day of a thirty day menstrual cycle (medium length menstrual cycle). For PT11-2 (FIG. 1C), day zero (0) on the x-axis represents the seventeenth day of a thirty-four day menstrual cycle (long menstrual cycle).

Still referring to FIGS. 1A through 1C, the figure illustrates normal vaginal CA125 level as a function of time-of-cycle for menstrual cycles where ovulation occurs. The plotted functions represent reproducible and cyclic patterns or signatures. The vaginal CA125 versus time of cycle is also called in this disclosure the day-specific vaginal CA125 signature or function. Suitably, uLH is the standard for identifying a menstrual cycle's day of ovulation since ovulation usually occurs twenty, plus or minus three hours after the first positive uLH with 95% confidence between the interval of fourteen and twenty-six hours. This said, the vaginal CA125 signature/day-specific vaginal CA125 function is not determined or restricted by the uLH indicator. Instead, the uLH indicator is merely used as a means to identify or index the days of a woman's menstrual cycle. In other words, other biomarkers of the menstrual cycle days can be used to index the vaginal CA125 signature or function without departing from the spirit and intent of this disclosure. For instance, other indicators of ovulation, such as basal body temperature, follicular rupture by ultrasound, serum LH, and ratio of urine estrone-3-glucuronide to urine pregnanediol-3-glucuronide, known in the art may be used to define the days in the vaginal CA125 signature or function. In other words, the form and vital attributes of the vaginal CA125 signature/day-specific vaginal CA125 function are preferably dependent only on the levels of vaginal CA125 over the menstrual cycle rather than the methods used to identify ovulation.

For each CA125 measurement, a sample of vaginal fluid must be collected and assayed over the course of a normal ovulatory cycle. Many apparatuses are available for collecting vaginal fluid. Preferred vaginal fluid collecting apparatuses create ease for the female user and increase the sensitivity and precision of measurements of CA125 using an antibody assay. In one preferable embodiment, a cotton-tipped swab, called a vaginal swab, is employed for collecting vaginal contents which includes vaginal fluid and vaginal epithelial. In one embodiment, the swab features: a plastic shaft of approximately 15 cm in length; and, a cotton-tipped head approximately 1.5 cm in length. This said, the collection apparatus need not be limited to a cotton swab. In one mode of operation, the vaginal swab may be inserted by the female user just beyond the introitus, for approximately two to three centimeters depth (the insertion depth being variable) so that the swab does not reach the cervix or distal vagina. Suitably, the collection procedure continues with a single rotary motion of the vaginal swab while inserted in the subject at the above described depth. The contents of a vaginal swab, which includes vaginal fluid and epithelial cells, may be transferred to the cotton head through an adsorption process during insertion and rotation of the swab.

It should be noted that vaginal CA125 levels and the day-specific vaginal CA125 signature/function are not affected by sexual intercourse and semen if the vaginal swab collection is applied more than 8 hours after sexual intercourse. The method is not necessarily restricted to this time interval between sexual intercourse and vaginal swab sampling. It should also be noted that the vaginal CA125 levels and the signature function are not restricted to or affected by a particular timing during the day for sampling.

Once a sample is collected, the cotton tip may be transferred to a vial containing 0.5 ml of deionized water wherein the cotton tip, with adsorbed substrate, is immersed in said deionized water. The tip may suitably be swirled in the deionized water for 3 seconds, removed from the deionized water, and compressed (i.e., wrung-out) on the vial side to collect further eluted specimen before discarding the spent cotton tip and swab.

Once collected in the vial, the sample is ready for CA125 assay. In a preferred embodiment, vaginal CA125 molecules will not only be collected from vaginal fluid, but also be released from vaginal epithelial cells, solubilized in water, rendered in an extended conformation, and be readily available for capture by monoclonal assay technology. The vaginal CA125 signature to identify fertile and infertile phases illustrated by FIG. 1 is obtained by measurement of CA125 levels with the IMMULITE OM-MA® kit and system according to the manufacturer's specifications over a menstrual cycle. The monoclonal antibody used in this assay is the OV185, which detects the M11 epitope of CA125. However, the method disclosed here is not limited to the OV185 antibody because any sensitive and specific monoclonal antibody to CA125 or other sensitive and specific modality would be reasonably expected to generate the diagnostic day-specific vaginal CA125 signature/function of this disclosure. The method disclosed here is based upon CA125 assay of the water containing solubilized vaginal swab contents, and therefore collection vials were centrifuged for one minute at ten-thousand revolutions per minute in a standard microfuge to separate particulate and cellular debris from the liquid.

The concentration of CA125 in the supernatant from the centrifuged collection vials represents the ‘vaginal CA125 level or concentration’ or ‘day-specific vaginal CA125 level or concentration’. It reflects the CA125 concentration in the original 0.5 ml volume of deionized water used to extract the adsorbed vaginal swab contents. The vaginal CA125 levels disclosed herein refer to the concentration of this ‘unit collection volume’ or ‘unit volume’ or ‘unit assay.’ It should be noted, however, the day-specific vaginal CA125 signature/function is not dependent upon a specific collection volume of deionized water. The 0.5 ml collection volume recited herein was selected do keep vaginal CA125 levels between 0 and 500 U/ml in order to optimize the sample for the commercial OM-MA Siemans IMMULITE 2000® immunoassay system. Where the vaginal CA125 concentrations surged to levels higher than 500 U/ml during the fertile and periovulatory phases, collection volumes may be diluted usually to 1:10 to obtain a CA125 measurement in the optimal range of 0 and 500 U/ml. When so diluted, the 0.5 ml ‘unit collection volume’ concentration can be established from the dilution factor. Furthermore, the day-specific vaginal CA125 signature/function is not suitably limited by the centrifugation separation because, in a preferred embodiment, measurements of vaginal CA125 levels involve transfer of the water, including solubilized vaginal swab contents, and cellular matter to filter paper, other absorbent membrane, or other platform for CA125 analysis.

Step two and three of the disclosed method are: collecting a swab of vaginal epithelial and fluid from the distal vagina near the vaginal orifice (distinct from cervical mucus) of the subject; and measuring the CA125 in the swab. These steps are accomplished in the same manner disclosed above for collecting and determining the vaginal CA125 signature or function.

Step four of the disclosed method is determining the time-of-cycle, that is, the phase of the cycle (period of potential fertility or days of maximum fertility or luteal/infertile days) using the woman's vaginal CA125 level. The vaginal CA125 levels corresponding to the day of cycle for a subject with a normal menstrual or ovulatory cycle can be used to predict the period of potential fertility. Ovulation occurs most frequently the day after the day of the uLH peak; that is, uLH peak=day 0, day after=day +1, day of putative ovulation. The period of potential fertility is generally thought to be the day of ovulation and the preceding five days; this is a six-day interval or period of potential fertility, indexed as day minus 4 (day −4) to day +1 of the cycle. The days of maximum fertility, generally considered to be the day of the first positive uLH (day 0) and the day preceding (day −1) in the period of potential fertility, may be assessed by the same method. However, it is important to note that the period of potential fertility is a maximum interval and the days of actual significant fertility may be considerably less depending upon the fecundity of cervical mucus. The days of high or maximum vaginal CA125 represent possible days of maximum fertility and may not always correspond to day −1 or day 0.

In one embodiment, vaginal CA125 levels of a subject on a given day may be referenced to the vaginal CA125 signature for the ovulatory normal female menstrual cycle. FIGS. 1A through 1C each represent vaginal CA125 signatures for a single ovulatory cycle of three different woman and, as a result, can be used to determine the time of cycle and fertility for a subject by comparison or algorithm.

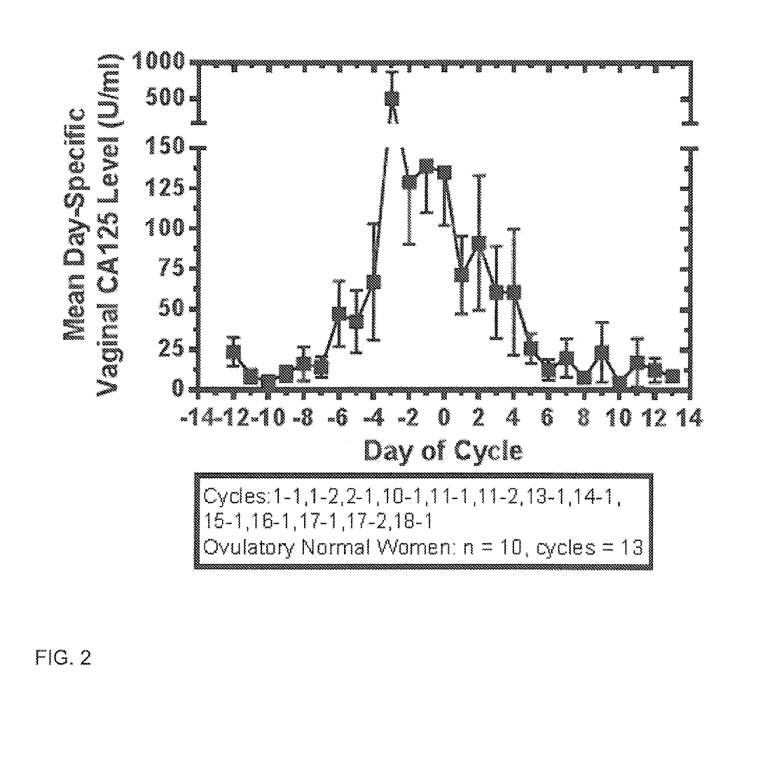

The accuracy of time of cycle or fertility predictions can be improved via averaging (a) the vaginal CA125 signatures of multiple women over a single menstrual cycle, (b) the vaginal CA125 signature of one woman over multiple menstrual cycles, or (c) the vaginal CA125 signatures of multiple women over multiple menstrual cycles. FIG. 2 shows the mean day-specific vaginal CA125 signature for thirteen ovulatory cycles by ten different ovulatory normal women. Ovulation was proven in these thirteen cycles by serum progesterone greater than three nanograms per milliliter after day zero (0) with a positive uLH. Still referring to FIG. 2, most of the sampling occurred immediately after the cessation of menses, but in some cycles, sampling was delayed by two days. The chart is useful for comparison to a subject's vaginal CA125 levels on a given day to determine fertility or time of cycle because:
i) a significant increase in vaginal CA125 level is observed with an inflection point by day negative four (−4) or five (−5) to signal the approach or start of the period of potential fertility;
ii) maximum fertility is observable at high levels of vaginal CA125 during days negative three (−3) to zero (0);
iii) an inflection point for a decrease in vaginal CA125 levels is observable during days zero (0) to positive two (+2) indicating an ending to fertility and;
iv) a return to low basal vaginal CA125 levels is observed at approximately days positive four (+4) to six (+6) indicating the infertile luteal phase of a woman's menstrual cycle.

Still referring to FIG. 2, it should be noted that a considerable amount of natural variation in the day of ovulation in the normal ovulatory female cycle is noticeable, and the actual day of ovulation may occur past day positive one (+1) or, in a minority of cases, prior to day zero (0), where day 0 is the first day of positive uLH. Such variations might explain the relatively high levels of vaginal CA125 that can occur up to day positive two (+2). A smaller spike in vaginal CA125 is sometimes observed during the early post-ovulatory phase (see, e.g, cycle 17-2, day positive four (+4), FIG. 1B) which correlates with a second peak in estrogen during the luteal phase.

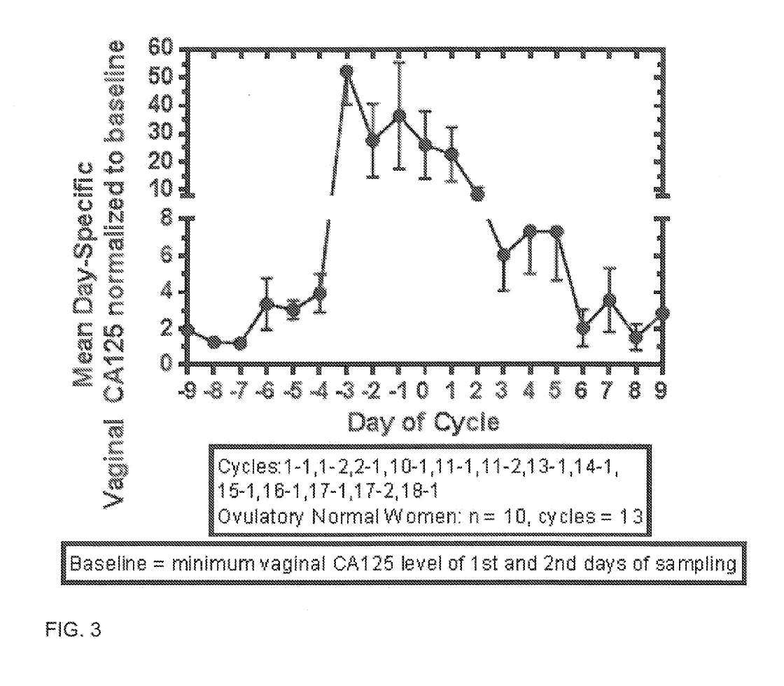

The accuracy and ease of observation of time of cycle or fertility predictions can be improved via normalization because: different women will inherently have different vaginal CA125 levels; and observations are made easier with a normalized baseline. FIG. 3 shows a plot of the mean normalized day-specific vaginal CA125 levels of the thirteen cycles of ten women of FIG. 2. For normalization, the level of every CA125 sample of every woman of the FIG. 2 plot was divided by the respective woman's lowest level of vaginal CA125 for the first two days of sampling to give a normalized baseline for each woman. The plot also includes indications of standard error. In one embodiment, this derived or normalized signature or function may be utilized for an algorithm to signal the period of potential fertility and the transition to the luteal (infertile) phase for a subject female. Other processed functions and algorithms are possible utilizing the day-specific vaginal CA125 signature and, as a result, disclosed methods of assessing fertility are not limited by the algorithm of FIG. 3. It should be noted that, in order to utilize the normalized vaginal CA125 signature or function to assess a subject's fertility in view of vaginal CA125 levels, the subject's vaginal CA125 level needs to be scaled (normalized), usually via dividing the vaginal CA125 level by a measurement of vaginal CA125 level on the first or second day after the end of menses.

Still referring to FIG. 3, when normalized the vaginal CA125 levels on the y-axis are without units and represent increases from the baseline of 1 (i.e., minimum normalized vaginal CA125 of the subject). As shown in FIG. 3, there is generally:
(i) a greater than or equal to three-fold increase in vaginal CA125 levels by day negative six (−6), which signals the period of potential fertility with sufficient warning for contraception purposes (e.g., abstinence during the period of potential fertility);
(ii) a greater than or equal to six-fold increase maximum (i.e., between 6.3 and 492 fold increase) to signal the maximum fertility of the subject (i.e., between day negative three (−3) to positive one (+1));
(iii) an inflection point with significant negative slope for vaginal CA125 levels during the interval of day positive one (+1) to positive three (+3) to signal transition to the infertile phase of the menstrual cycle; and,
(iv) a return to baseline vaginal CA125 levels generally by day positive six (+6) and following for predicting the infertile phase of the menstrual cycle.

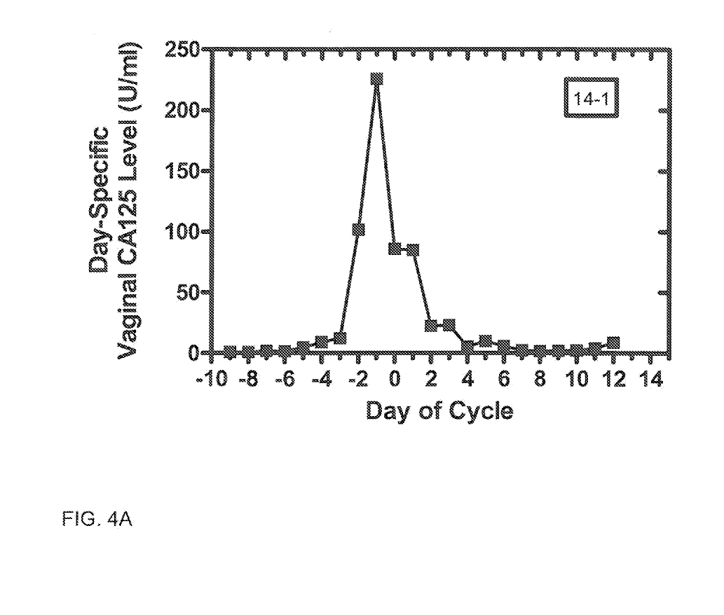

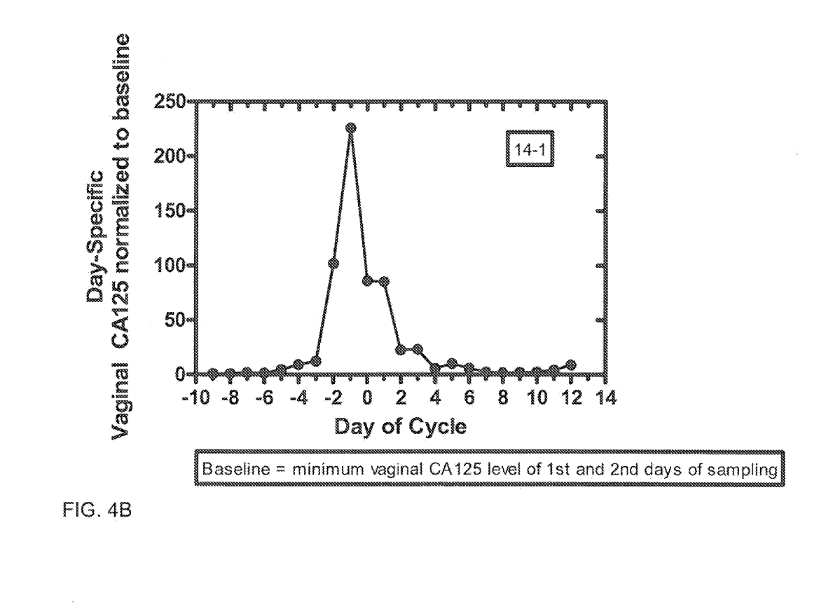

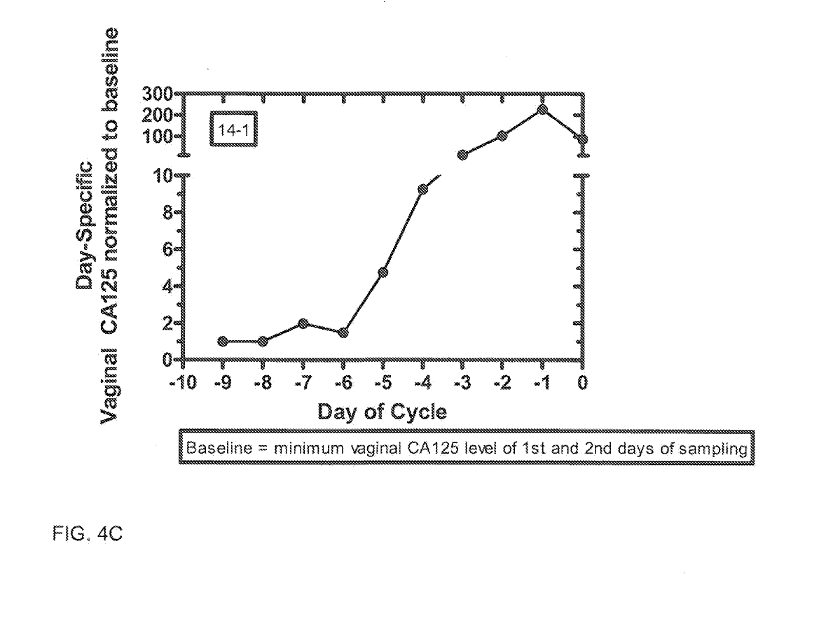

As alluded to above, the signature curve for day-specific vaginal CA125 shown in FIGS. 1A through 1C, 2, and 4A can be used to create algorithms to signal the period of potential fertility and other phases of the menstrual cycle. This is demonstrated, for example, by comparing FIGS. 4A with FIGS. 4B and 4C. For FIG. 4A, the woman, PT14, cycle 14-1, provided vaginal samples for measurement of CA125 starting the day after menses stopped and continued throughout the luteal (post-ovulatory) phase. PT14's vaginal CA125 signature plot was created by setting the day of the first positive uLH to day zero (0). FIG. 4B is a normalized plot of the vaginal CA125 signature of the woman PT14. The plot was created via dividing all of the vaginal CA125 measurements by the lowest CA125 measurement from the first two days of sampling. It should be noted that, for the woman PT14 during cycle PT14-1, the baseline CA125 level was less than or equal to 1 U/ml and therefore the day-specific vaginal CA125 plot (FIG. 4A) and derived (normalized) function (FIG. 4B) appear almost identical. FIG. 4C is a magnified view of the interval of day negative nine (−9) to day zero (0) of FIG. 4B, which includes the period of potential fertility.

The derived (normalized) vaginal CA125 plot or signature of FIG. 3 may result in an algorithm for predicting the start of the period of potential fertility for birth control purposes by comparison with FIG. 4B or 4C. As shown, the greater than three-fold increase in vaginal CA125 levels, which signals the start of the potential period of fertility in the women of FIG. 3, is manifest by the woman of FIGS. 4B and 4C at day negative five (−5). This means that the woman of FIGS. 4B and 4C may initiate abstinence at that time for contraception. FIGS. 4B and 4C show a greater than 4.7-fold normalized vaginal CA125 level by day negative five (−5), a peak of 226-fold normalized vaginal CA125 on day negative one (−1), and a significant fall in normalized vaginal CA125 levels by day positive two (+2). When compared with FIG. 3, the timing for abstinence for birth control purposes for cycle PT14-1 shown in FIGS. 4A through 4C is day minus five (−5) to day positive two (+2) (abstinence interval), and for fertility enhancement days negative two (−2) through positive one (+1) (days for sexual intercourse).

It should be noted that comparison of FIG. 3 and FIGS. 4A through 4C are exemplary only. Other derived functions of the vaginal CA125 signature are possible, and may lead to a similar or different start day for the period of potential fertility and transition day to the luteal or infertile phase. The days/interval of abstinence and probability of avoidance of conception, and conversely, the days of sexual intercourse and probability of conception can be modulated through different analytical processing of the day-specific vaginal CA125 function. Any derived analytic function of the vaginal CA125 signature follows from the disclosure herein, the basis of any such analytic function to establish the period of potential fertility, days of maximum fertility, or transition to the post-luteal (infertile phase), being the day-specific vaginal CA125 signature or function.

TABLE 1. Provides fertility metrics determined by the preferable vaginal CA125 method in thirteen total ovulatory cycles from ten ovulatory normal women compared with conventional natural family planning—the Ovulation Method. Table 1. compares the start of the potential period of fertility as signaled by the disclosed method to the Ovulation Method (OM) in conventional natural family planning. Traditional natural family planning, which relies on subjective characteristics of cervical mucus/vaginal fluid to identify the potential period of fertility, was in the majority of cases either: more restrictive (the interval of abstinence increased relative to the preferable method) or less effective (days for ‘safe’ sexual intercourse occurring more frequently within the period of potential fertility compared to the preferable method) or identified day zero (0) (day of positive uLH)—one of the presumably most fertile days—as infertile. In summary, natural family planning (OM) was similar to the disclosed method in one of twelve cycles, with the disclosed method being superior in eleven of twelve cycles. In view of Table 1, the method discloses a superior technology to identify the potentially fertile and the infertile phases of the ovulatory cycle.

In Table 1:

a super script “a” notes
Ovulation Method of conventional natural family planning (NFP) utilizes subjective sensation of vaginal wetness for fertile days and dryness for infertile days;
   a super script “1” notes
Days are indexed to ‘Day 0’, day of positive urine luteinizing hormone. These cycles are ovulatory as determined by a rise in serum progesterone to ≧3 ng/ml sometime after Day 0. Potential Period of Fertility (PPF) is considered a 6-day interval (−4 to +1). The time of maximal fertility is generally on day −1 and day 0. The signal for the start of the PPF utilizing the invention here is a 3-fold increase in vaginal CA125 above baseline (minimum level of first or second days of vaginal sampling); however, the invention is not constrained by this algorithm, and other discriminant functions with this invention for birth control are possible to vary effectiveness (to reduce probability of pregnancy) and restrictiveness (to reduce number of days of abstinence).
   a super script “2” notes
Day of maximum (Peak) vaginal CA125 is during the interval, pre-ovulatory phase which includes the PPF and until day +1.
   a super script “3” notes
Peak vaginal CA125 level (U/ml) is determined utilizing the invention. All Peak vaginal CA125 levels were ≧26-fold baseline.
   a super script “4” notes
Days of increased fertility are defined as days of vaginal CA125 ≧6-fold baseline. These are days of predicted increased fertility utilizing the invention, but all days of possible fertility (PPF) by the invention would extend from the start day of PPF to the day past day +1 or days after the rise in vaginal CA125.
   a super script “5” notes
Start day of PPF by conventional NFP (Ovulation Method) is the start day of vaginal wetness.
   super scripts “6” through “10” note
The standard for the PPF is generally taken as −4 to +1, where Day 0 is the day of the positive urine LH. The day of maximum fertility is generally taken as day −1 and day 0. Conventional NFP is more restrictive (for birth control) if its metric signals more days of abstinence before day −4 compared to the invention and less effective (for birth control) if its metric signals the start of the PPF after that signaled by the invention.
The invention was superior to convention NFP/Ovulation Method in determining the PPF; Conventional NFP was only more effective for birth control than the invention in one cycle (15-1). In addition, conventional NFP determined some days of maximal fertility (Day 0) as infertile dry days, indicating the invention is not only more effective for birth control, but also for fertility enhancement.

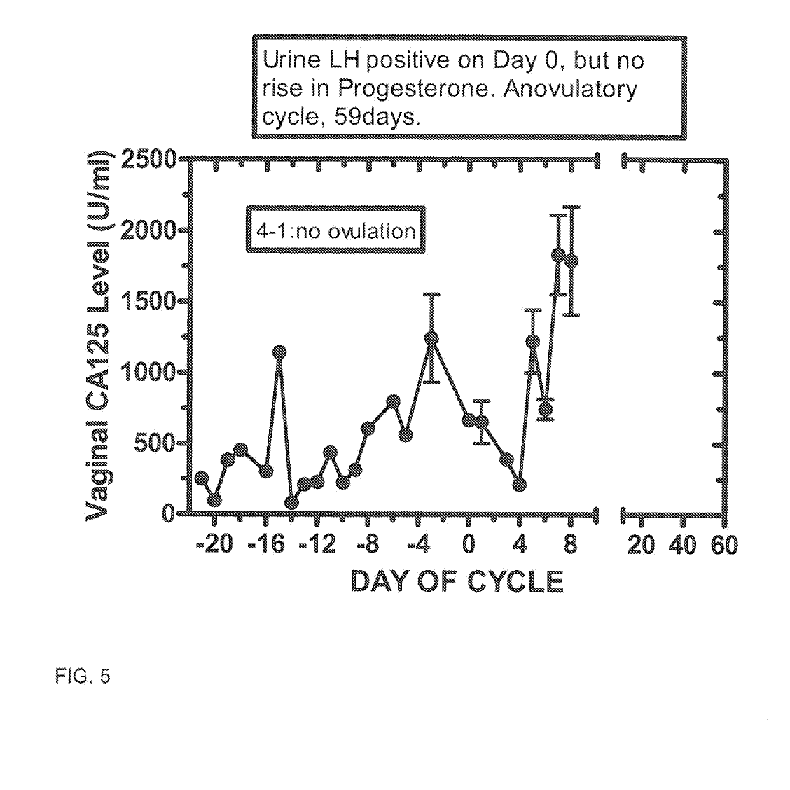

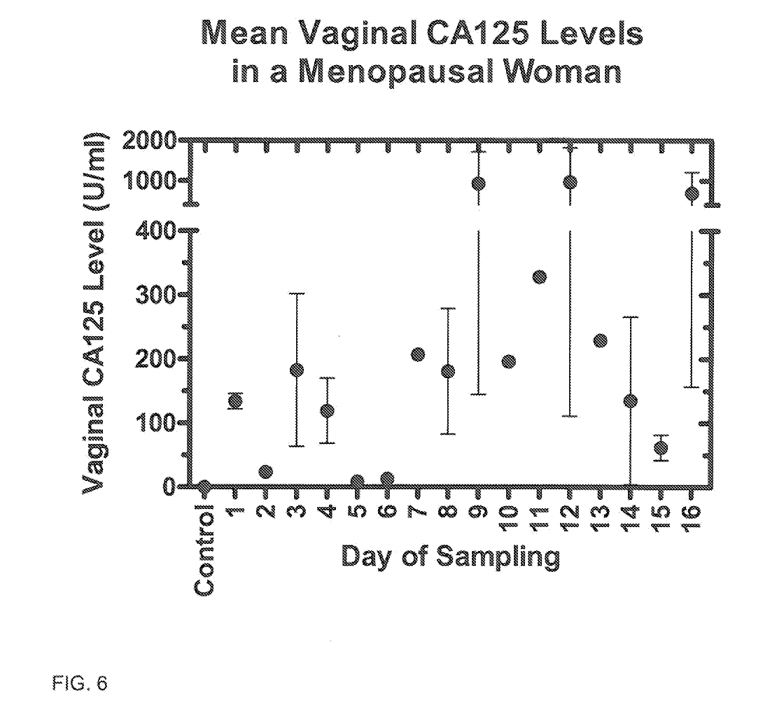

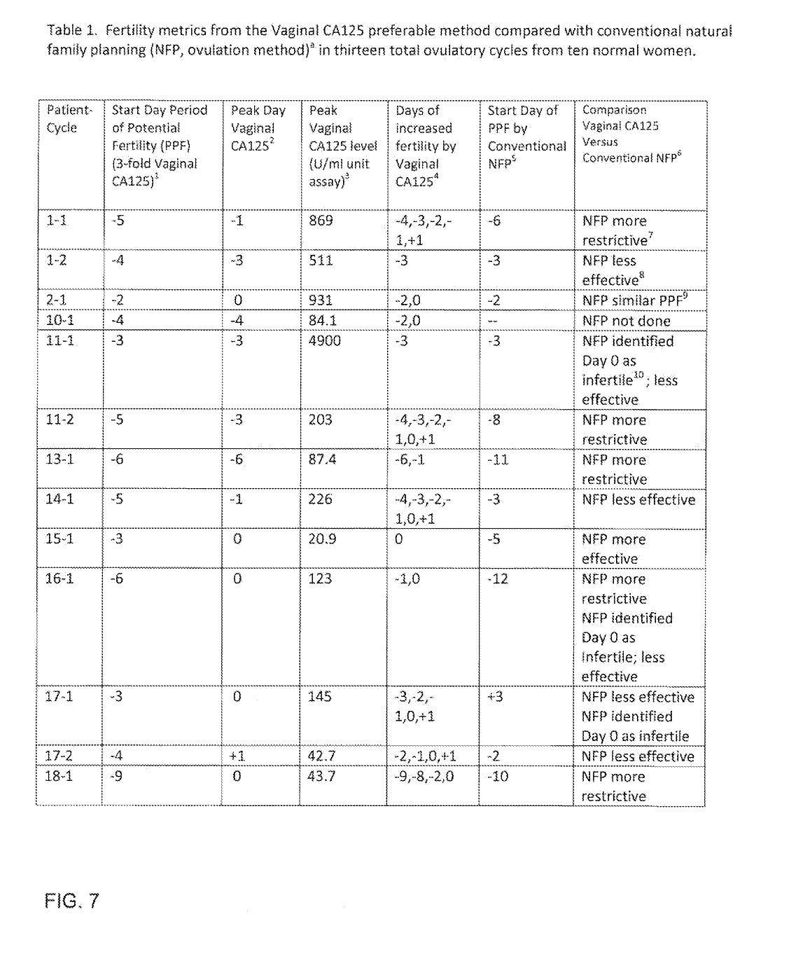

The establishment by this disclosure of a new marker of normalcy—the day-specific vaginal CA125 signature—allows for the discovery of female reproductive variation, abnormality, and pathology. A preferable embodiment of this disclosure is the day-specific vaginal CA125 signature as a diagnostic tool in the field of gynecologic and female reproductive endocrinology. FIGS. 5 and 6 are examples of abnormalities that may be diagnosed via the CA125 signature or profile.

FIG. 5 illustrates a cycle of fifty nine days where urine LH was positive on day 31 of the cycle, a dramatically delayed apparent ovulation by the urine LH method. However, this was an anovulatory cycle or at least ‘insufficient’ cycle, which was indicated by the serum progesterone levels remaining at 0.2 ng/ml nine days after the urine LH peak. In this progesterone-deprived anovulatory cycle, the absolute levels of vaginal CA125 were above 500 U/ml for numerous days, atypical for ovulatory normal women (compare with, e.g., FIG. 2). Thus, both an abnormal pattern of vaginal CA125 and abnormal levels identify progesterone-deprivation and anovulation.

FIG. 6 shows the vaginal CA125 profile for a menopausal woman. No ovulation or cyclicity of estrogens or progesterone occurs during menopause. As shown, two separate series of vaginal CA125 collections and measurements were combined, and the measurements plotted by day of sampling. In the plot, day positive one (+1) was arbitrarily assigned the start day for both series of samplings. In one series, sampling occurred daily, in the other series sampling was not daily. In the case of the menopausal woman of FIG. 6, there is both estrogen and progesterone deprivation due to ovarian failure; again, no cyclic pattern occurs and the vaginal CA125 are on average higher than for the ovulatory normal cycle.

The method disclosed is not limited to diagnosis of anovulatory and low estrogen or low progesterone states. This method extends to diagnosis of any gynecologic or reproductive pathology based upon perturbation of vaginal CA125, either abnormalities of vaginal CA125 signature or absolute vaginal CA125 levels. Those familiar with the field would expect abnormalities of CA125 levels and signature in cancerous states such as ovarian, endometrial, and vaginal neoplasms.

Furthermore, the method disclosed here can be combined with devices available in the present art and science of fertility detection to devise further birth control and fertility methodologies. For example, although the method disclosure here is a ‘stand-alone’ method for the prediction of the period of potential fertility and the ovulatory, and luteal phases for pregnancy avoidance or enhancement, the method disclosed here can be combined with the available uLH Clearblue Kit® wherein a positive uLH provided by the Clearblue kit would be a ‘double-check’ method to mark the prospective luteal phase. However, the uLH Clearblue Kit® cannot signal the start of the period of potential fertility and therefore cannot be used for birth control purposes as a stand-alone method, and in addition, does not identify days of potentially maximum fertility.

In one embodiment, a kit may be provided for collecting samples from women subjects. In one embodiment, the kit may include a cotton swab as described above and a vial of deionized water of appropriate volume for assay. Suitably, a subject may introduce the swab into her own vagina and collect a specimen, swirl the specimen in the vial of deionized water as described above, and deliver the vial to a laboratory for measurement of CA125 levels or measure the CA125 level with a future home CA125 assay kit.

In one specific mode of operation, the method is accomplished by:
(1) obtaining a cotton tipped applicator;
(2) on the first or second day after a subject's menses, introducing the applicator to into the vagina of a subject at a short distance from the orifice;
(3) rotating the applicator to obtain a swab of vaginal fluid (Note: the best time for the swab is in the morning but the vaginal swab should not preferably take place within 8 hours of sexual intercourse);
(4) removing the swab and immersing the same in 0.5 ml of deionized water (in a 1.5 ml microfuge tube) and swirled for 3 seconds whereafter the swab is squeezed to remove water from the cotton;
(5) discarding the swab and centrifuging the sample to remove cellular debris and the supernatant before submitting the same for OM-MA assay on a Siemans Immulite 2000 machine or similar assay platform to determine the “day-specific vaginal CA125 level”;
(6) the day specific CA125 level is used to calculate fertility according to a predetermined algorithm.
   For example, start day of fertility is the day when the vaginal CA125 level divided by the initial minimum vaginal CA125 level is equal to 3.0 or higher, the day of peak fertility is the day when the vaginal CA125 level divided by the initial minimum vaginal CA125 level is equal to 6.0 or higher, and the day of luteal phase is the day when the vaginal CA125 level divided by the initial minimum vaginal CA125 level is equal to a baseline of approximately 1.0. Chronic levels of high CA125 are indicative of ovarian failure and lack of progesterone.

This specification and the appended figures illustrate only typical embodiments or principles disclosed in this application, and therefore, are not to be considered limiting of its scope, for the invention may admit to other equally effective embodiments that will be appreciated by those reasonably skilled in the relevant arts. Any invention disclosed by this specification is defined by the claims. While the ideas herein disclosed have been described by means of specific embodiments and applications thereof, numerous modifications and variations could be made thereto by those skilled in the art without departing from the scope of the invention set forth in the claims.

We claim:

1. A method of assessing fertility in a human female with a fertility kit comprising the steps of:
providing a kit with a swabbing and collection means for vaginal fluids;
using said swabbing and collection means from the kit to collect a sample of vaginal fluid;
establishing a vaginal CA125 signature for the female, wherein the vaginal CA signature is defined by a plot of vaginal CA125 levels of the female measured from samples collected from the kit versus day of menstrual cycle for the female;
identifying an indication of fertility on the vaginal CA125 signature

2. The method of claim 1 wherein the step of identifying an indication of fertility is accomplished by:
identifying a pre-ovulatory baseline vaginal CA125 level; and,
identifying the start of the period of potential fertility by locating a day of the menstrual cycle on the plot that is associated with a vaginal CA125 level that is approximately three times greater than the base line vaginal CA125 level.

3. The method of claim 1 wherein the step of identifying an indication of maximum fertility is accomplished by:
identifying a pre-ovulatory baseline vaginal CA125 level; and,
identifying the days of maximum fertility by locating the days of the menstrual cycle on the plot that are associated with vaginal CA125 levels that are approximately six times or greater than the baseline vaginal CA125 level.

4. The method of claim 1 wherein the step of identifying an indication of the transition to the infertile phase by:
identifying a baseline CA125 level; and,
identifying the start of infertile/luteal phase by locating the days of the menstrual cycle on the plot that are associated with a baseline CA125 level after the vaginal CA125 peak.

5. The method of claim 1 wherein the step of establishing a vaginal CA125 signature is accomplished by:
collecting samples of vaginal fluid and epithelial over the course of the females menstrual cycle;
establishing the day of ovulation by determining the first day of positive urine luteinizing hormone;
assaying the samples to measure the concentration of vaginal CA125 in the sample; and,
plotting the vaginal CA125 concentration verses the day of menstrual cycle wherein day zero is the day established by determining the first day of positive urine luteinizing hormone.

6. The method of claim 5 wherein samples of vaginal fluid are collected via insertion of a cotton swab into the vagina of the female.

7. The method of claim 6 wherein the cotton swab is defined by a shaft and a cotton-tipped head

8. The method of claim 7 wherein the shaft is approximately fifteen centimeters in length and the cotton-tipped head is approximately one and a half centimeters in length.

9. The method of claim 7 wherein insertion of a cotton swab into the vagina of the female is for approximately two to three centimeters depth.

10. The method of claim 7 wherein the insertion of the cotton swab into the female's vagina does not reach the cervix or distal vagina.

11. The method of claim 7 wherein insertion of the cotton swab is not accomplished within eight hours of intercourse by the female.

12. A method of contraception in a human female comprising the steps of:
establishing a vaginal CA125 signature for the female, wherein the vaginal CA125 signature is defined by a plot of vaginal CA125 levels of the female versus day of menstrual cycle for the female;
identifying the period of potential fertility on the vaginal CA125 signature; and,
directing the female to abstain from sexual intercourse over the period of potential fertility.

13. The method of claim 12 wherein the step of establishing a vaginal CA125 signature is accomplished by:
collecting samples of vaginal fluid and epithelial over the course of the females menstrual cycle;
establishing the day of ovulation by determining the first day of positive urine luteinizing hormone;
assaying the samples to measure the concentration of vaginal CA125 in the sample; and,
plotting the vaginal CA125 concentration verses the day of menstrual cycle wherein day zero is the day established by determining the first day of positive urine luteinizing hormone.

14. The method of claim 13 wherein samples of vaginal fluid are collected via insertion of a cotton swab into the vagina of the female.

15. The method of claim 14 wherein the cotton swab is defined by a shaft and a cotton-tipped head

16. The method of claim 15 wherein the shaft is approximately fifteen centimeters in length and the cotton-tipped head is approximately one and a half centimeters in length.

17. The method of claim 15 wherein insertion of a cotton swab into the vagina of the female is for approximately two to three centimeters depth.

18. The method of claim 15 wherein the insertion of the cotton swab into the female's vagina does not reach the cervix or distal vagina.

19. The method of claim 15 wherein insertion of the cotton swab is not accomplished within eight hours of intercourse by the female.
